(19) United States
(12) Patent Application Publication
(10) Pub. No.: US 2006/0269935 A1
(43) Pub. Date: Nov. 30, 2006

(54) Genetic predictor for clinical use of drugs used in the treatment of neurological conditions

(75) Inventors: Sarah K. Tate, London (GB); Gianpiero L. Cavalleri, London (GB); Stephanie Schorge, London (GB); Sanjay Sisodiya, Bucks (GB); Nicholas W. Wood, London (GB); David B. Goldstein, London (GB)

Correspondence Address: Fulbright & Jaworski L.L.P., Fulbright Tower, 1301 McKinney, Suite 5100, Houston, TX, 77010-3095, US

(73) Assignee: UCL Biomedica PLC, London (GB)

(21) Appl. No.: 11/377,985

(22) Filed: Mar. 17, 2006

Related U.S. Application Data
(60) Provisional application No. 60/663,413, filed on Mar. 18, 2005.

(30) Foreign Application Priority Data
Sep. 27, 2005 (AU) 2005215928

(51) Int. Cl. C12Q 1/68

(52) U.S. Cl. 435/6

(57) ABSTRACT

A method for determining the dosage regime of a drug suitable for use in the treatment of a neurological condition in a subject, which method comprises typing the SCN1A gene of the subject. The method may be used to determine the dosage regime of an anti-epileptic drug (AED) in a subject. A subject may be treated in accordance with the dosage regime determined using such a method.

This application claims priority to U.S. Provisional Patent Application No. 60/663,413, filed Mar. 18, 2005, and to Australian Patent Application No. 2005215928, filed Sep. 27, 2005, both of which are incorporated by reference herein in their entirety.

### FIELD OF THE INVENTION

The invention relates to methods for determining the dosage regime of a drug in the treatment of a neurological condition, in particular epilepsy, and to methods for treating a subject accordingly.

### BACKGROUND TO THE INVENTION

Phenytoin and carbamazepine are important first-line AEDs widely prescribed throughout the world. Control of epilepsy with phenytoin can be a difficult and lengthy process due to the drug's narrow therapeutic index and the wide inter-individual range of doses required. Similarly, appropriate doses for carbamazepine take time to determine because of autoinduction of metabolism and neurologic side effects generally assumed to necessitate slow dose increases. Adverse drug reactions (ADRs) are relatively common for both drugs.

### SUMMARY OF THE INVENTION

This invention is based on the identification of a significant association of an intronic polymorphism in the sodium channel, voltage-gated, type I, alpha (SCN1A) gene with maximum doses of both carbamazepine and phenytoin (P=0.0051 and P=0.014). The inventors have shown that this polymorphism disrupts the consensus sequence of the 5′ splice donor site of a highly conserved alternative exon (5N) and that it significantly affects the proportions of the alternative transcripts in individuals with a history of epilepsy. Analysis of transcript levels in individuals without a history of epilepsy shows no such effect. In addition, the inventors have shown that a known functional polymorphism in CYP2C9 also shows highly significant association with maximum dose of phenytoin (P=0.0066). These results provide the first clear evidence of a drug target polymorphism associated with the clinical use of AEDs.

In accordance with the present invention there is thus provided a method for determining the dosage regime of a drug suitable for use in the treatment of a neurological condition in a subject which method comprises typing the SCN1A gene of the subject.

The invention also provides:
a test kit suitable for use in a method for determining the dosage regime of a drug suitable for use in the treatment of a neurological condition in a subject, which test kit comprises means for typing the SCN1A gene of the subject;
a method for the treatment or prophylaxis of a neurological condition in a subject, which method comprises:
   (a) determining the dosage regime of a drug suitable for use in the treatment of the neurological condition using the method set out above; and
   (b) administering a therapeutically effective amount of the drug to the subject in accordance with the dosage regime determined in (a).
products containing:
   (i) means for typing the SCN1A gene of a subject; and
   (ii) a drug suitable for use in the treatment of a neurological condition as a combined preparation for simultaneous, separate or sequential use in a method of treatment of the human or animal body by therapy.

### BRIEF DESCRIPTION OF THE FIGURES

FIG. 1 shows the distribution of maximum phenytoin doses.

FIG. 2 shows the distribution of maximum carbamazepine doses.

FIG. 3 shows the distribution of maximuim carbamazepine doses (mg) for each SCN1A IVS5-91 G>A genotype.

FIG. 4a shows the genomic structure of SCN1A surrounding exons 5N and 5A and regulation of exon 5N in epileptic tissues.

FIG. 4b shows the proportion of SCN1A 5N transcript in brain tissue from patients with a history of epilepsy.

FIG. 5 shows the association of 20 unlinked markers with maximum carbamazepine dose.

### BRIEF DESCRIPTION OF THE SEQUENCE LISTING

SEQ ID NO: 1 sets out the complete nucleotide sequence of exon 5N.

SEQ ID NO: 2 sets out the complete amino acid sequence of exon 5N

SEQ ID NO: 3 sets out the complete nucleotide sequence of exon 5A.

SEQ ID NO: 4 sets out the complete amino acid sequence of exon 5A.

SEQ ID NO: 5 sets out the sequence of a forward oligonucleotide primer used to amplify exons 5A and 5N using RT-PCR.

SEQ ID NO: 6 sets out the sequence of a reverse oligonucleotide primer used to amplify exons 5A and 5N using RT-PCR.

### DETAILED DESCRIPTION OF THE INVENTION

Throughout this specification, the word “comprise”, or variations such as “comprised” or “comprising”, will be understood to imply the inclusion of a stated element, integer or step, or group of elements, integers or steps, but not the exclusion of any other element, integer or step, or group of elements, integers or steps.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

Phenytoin and carbamazepine are effective and inexpensive AEDs widely prescribed throughout the world. Control of epilepsy with phenytoin and carbamazepine can be a difficult and lengthy process because, as with many AEDs, a broad range of doses is used, with the final “maintenance” dose normally determined by trial and error. Adverse drug reactions (ADRs) are relatively common for both drugs.

Phenytoin is metabolised by the hepatic cytochrome P450 enzymes CYP2C9 and CYP2C19, is transported by P-glycoprotein and targets the alpha subunit of the sodium channel. CYP2C9 is estimated to be responsible for up to 90% of phenytoin inactivation (Kupfer and Preisig, Eur. J. Clin. Pharmacol. 26, 753-759, 1984).

Substantial in vitro data demonstrate that both the *2 and *3 alleles (http://www.imm.ki.se/CYPalleles/. Human Cytochrome P450 (CYP) Allele Nomenclature Committee. 8-9-0004.) result in significant reductions in the metabolism of various CYP2C9 substrates, with *3 showing consistently greater reductions in intrinsic clearance than *2. There have been numerous reports on phenytoin pharmacokinetics (see Table 1 below), but no large studies of response.

TABLE 1
Functional effects of CYP2C9*2 and *3 polymorphisms

| CYP2C9 | | | |
|---|---|---|---|
| allele | In vitro expression | Pharmacokinetic | Mechanism |
| *2 | 29% reduction in | *2 carriers have increased serum | *3 mutation is located in substrate |
| | phenytoin | concentrations of phenytoin, following a | recognition site (SRS) 5, |
| | clearance compared | single dose in healthy volunteers(30) | accounting for reductions in |
| | to *1(29) | | binding capacity and intrinsic |
| | | | clearance(31). |
| *3 | 93 to 95% | *3 carriers have significantly lower | *2 allele is not located in an SRS. |
| | reduction in | maximal elimination rates than *1/1 | Mechanism responsible for |
| | phenytoin | patients(33, 34) and increased serum | reduction of metabolism rate is |
| | clearance compared | concentrations of phenytoin, following a | unclear(36). |
| | to *1(29, 32) | single dose in healthy volunteers(35) | |

Phenytoin acts by blocking voltage-sensitive sodium channels in neurons, and binds to the alpha subunit encoded by the brain-expressed genes SCN1A, 2A, 3A, and 8A. SCN1A is implicated in many Mendelian forms of epilepsy (Ceulemans et al., Pediatr. Neurol. 30, 236-243, 2004).

Also, passage of phenytoin across the blood-brain barrier is probably affected by P-glycoprotein. The ABCB1 gene carries a silent polymorphism in exon 26 (3435C>T, or rs1045642) that has been associated with altered expression levels of P-glycoprotein (Hoffmeyer et al., Proc. Natl. Acad. Sci. USA 97, 3473-3478, 2000) and also with a range of drug responses and clinical conditions (Soranzo et al., Genome Res. 14, 1333-1344, 2004). In particular, this polymorphism has been weakly correlated with both response to AEDs (Siddiqui et al., N. Engl. J. Med. 348, 1442-1448, 2001) and phenytoin plasma levels (Kerb et al., Pharmacogenomics J. 1, 204-210, 2001). Although there is evidence that 3435C>T may not be causal, it is likely to be a marker for one or more causal variants (Soranzo et al., 2004, supra).

Carbamazepine is metabolised by the hepatic cytochrome P450 enzyme CYP3A4. Carbamazepine also induces CYP3A4 via activation of the pregnane X receptor (PXR, or NR112). This may contribute to the requirement of dose being increased over time after the drug is initiated. There is great interindividual variability in CYP3A4 expression and activity has been shown to vary up to at least 20-fold in vivo (Wilkinson, J. Pharmacokinet. Biopharm. 24, 204-210, 2001). Overall, it is not thought likely that variation in the CYP3A4 gene itself (either coding or regulatory) is a primary contributor to inter-individual variability in CYP3A4 enzyme activity (see, for example, Ozdemir et al., Pharmacogenetics 10, 373-388, 2000). We have therefore not included CYP3A4 in this study.

Carbamazepine also acts by binding to the alpha subunit of voltage-sensitive sodium channels in neurons, blocking high frequency discharges, and is a possible substrate of P-glycoprotein (Potschka et al., Neuroreport 12, 3557-3560, 2001). We have therefore associated variation in both SCN1A and ABCB1 with dosing of carbamazepine.

The inventors have shown that there is a significant association of an intronic polymorphism in the SCN1A gene with maximum doses of both carbamazepine and phenytoin (P=0.0051 and P=0.014). The inventors have shown that this polymorphism disrupts the consensus sequence of the 5′ splice donor site of a highly conserved alternative exon (5N) and that it significantly affects the proportions of the alternative transcripts in individuals with a history of epilepsy. Analysis of transcript levels in individuals without a history of epilepsy shows no such effect. In addition, the inventors have shown that a known functional polymorphism in CYP2C9 also shows highly significant association with maximum dose of phenytoin (P=0.0066).

Accordingly, the present invention provides a method for determining the dosage regime for a drug suitable for use in the treatment of a neurological condition in a subject. The method comprises typing the SCN1A gene of the subject. The dosage regime for treating the neurological condition in the subject may thereby be determined. That is to say, typing of the SCN1A gene of the subject will allow the dosage regime of the drug to be determined.

The subject may be one which is know to be suffering from a neurological condition or one suspected to be suffering from a neurological condition. The subject may be asymptomatic for the neurological condition. Thus, the drug may be intended for prophylactic use. The subject will typically be a mammal, for example a human.

Typing of the SCN1A gene typically comprises the measurement of any suitable characteristic of the SCN1A gene to determine the dosage regime of a drug suitable for use in the treatment of a neurological condition. In this context, the term “gene” encompasses not only the SCN1A coding sequence, but also untranslated, for example intronic sequences, and regulatory regions situated 5′ and 3′ to the coding sequences.

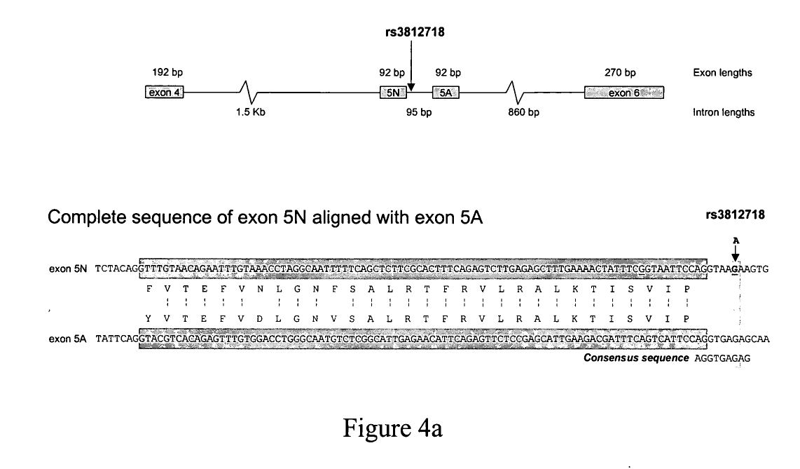

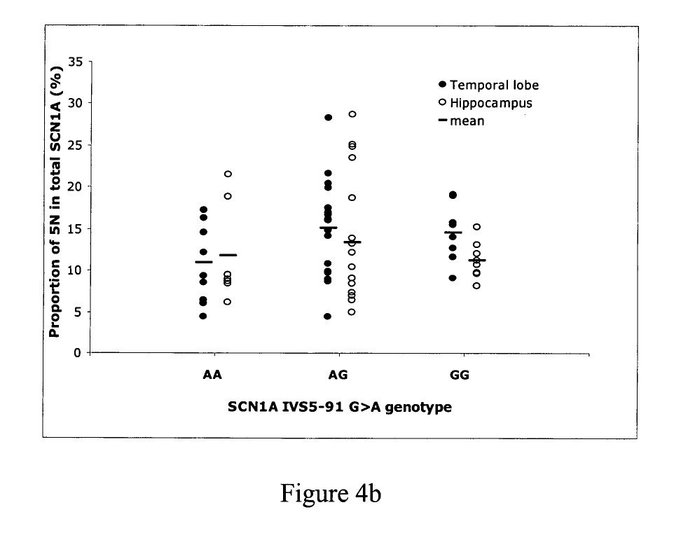

Suitable typing may involve determining whether the SCN1A gene of the subject comprises a polymorphism which is indicative of the dosage regime that should be used to treat the epilepsy. Such typing will typically involve determining the sequence of all or part of the SCN1A gene in the subject. Typically, a sequence encompassing the 5′ splice donor site of the 5N alternatively spliced exon may be determined. The sequence of the human SCN1A gene is set out in GenBank accession no. NM_{—}006920 (also see Rhodes et al., Proc. Natl. Acad. Sci. USA 101(30), 11147-11152, 2004). The region encompassing the 5′ splice donor site of the 5N alternatively spliced exon is set out in FIG. 4a (SEQ ID NO: 1) which corresponds to the sequence set out in SEQ ID NO: 1. Thus, typing according to the invention may involve determining all of part of that sequence in a subject or an allelic variant thereof.

The method of the invention may be carried out in vivo, although typically it is more convenient to carry the method out in vitro or ex vivo using a sample derived from the subject. The sample typically comprises a body fluid of the individual and may for example be obtained using a swab, such as a mouth swab. The sample may be a blood, urine, saliva, cheek cell or hair root sample. Alternatively, the sample may be brain tissue.

The sample is typically processed before the method is carried out, for example nucleic acid extraction, such as extraction of genomic DNA or RNA, in particular mRNA may be carried out. RNA, such as mRNA, extracted from a sample may subsequently be converted into cDNA. The nucleic acid or protein in the sample may be cleaved either physically or chemically (e.g. using a suitable enzyme, such as a restriction endonuclease). The polynucleotide in the sample may be copied (or amplified), e.g. by cloning or using a PCR based method.

In a preferred embodiment of the method of the invention, the typing comprises determining whether or not the subject has a polymorphism which is associated with the response to the drug which is suitable for use in the treatment of a neurological condition in the subject. Alternatively, such typing may involve determining whether the subject has a polymorphism which is in linkage disequilibrium with such a polymorphism.

Polymorphisms which are in linkage disequilibrium with each other in a population are typically found together on the same chromosome. Typically one is found at least 30% of the times, for example at least 40%, at least 50%, at least 70% or at least 90%, of the time the other is found on a particular chromosome in individuals in the population.

Thus a polymorphism which is not a functional susceptibility polymorphism, but is in linkage disequilibrium with a functional polymorphism, may act as a marker indicating the presence of the functional polymorphism.

Polymorphisms which are in linkage disequilibrium with any of the polymorphisms mentioned herein are typically located within 500 kb, preferably within 400 kb, within 200 kb, within 100 kb, within 50 kb, within 10 kb, within 5 kb, within 1 kb, within 500 bp, within 100 bp, within 50 bp or within 10 bp of the polymorphism. Typing of such a polymorphism is considered to be encompassed within the phrase “typing of the SCN1A gene” for the purposes of this invention

The polymorphism is typically an insertion, deletion or substitution with a length of at least 1, 2, 3, 4, 5, 10, 15 or more base pairs or amino acids. However, preferred polymorphisms are those where substitution of 1 base pair occurs, i.e. a single nucleotide polymorphism (SNP) is preferred. The polymorphism may be located 5′ to a coding region, in a coding region, in an intron or 3′ to a coding region.

The polymorphism which is detected is typically a functional mutation which is associated with the response to the drug which is to be used to treat the subject, but may be a polymorphism which is in linkage disequilibrium with a functional mutation, i.e. the polymorphism which is detected may be a marker for a functional mutation.

Generally, the polymorphism will be associated with the response to the drug used to treat the subject, for example as can be determined in a case/control study (e.g. as discussed in the Examples below). The polymorphism may cause a change in any characteristic of the SCN1A gene or protein, for example expression levels, such as transcription and/or translation rates, alternative splicing, degradation, enzymic activity, expression of a variant, cellular localisation or the pattern of expression in different tissues. The action of the drug may be mediated wholly or in part by the SCN1A gene or the product thereof and/or may be mediated directly or indirectly by the SCN1A gene or the product thereof.

Typically, the polymorphism is a single nucleotide polymorphism. A preferred polymorphism is that at position 104 of SEQ ID NO: 1. This SNP is known as rs3812718 (or IVS5-91 G>A or SNP7 in Weale et al., Am. J. Hum. Genet. 73, 551-565, 2003). The preferred polymorphisms may also be defined with reference to FIG. 4a, which sets out the same sequence as SEQ ID NO: 1. The polymorphism may also be defined with reference to the Homo sapiens chromosome 2 genomic contig NT_{—}005403 at position 17118961 (chromosome position 166735051) or by reference to the Homo sapiens chromosome 2 genomic contig, alternate assembly NT_{—}086633 at position 33891694 (chromosome position 157906503).

Other preferred polymorphisms are those at a position corresponding to position 104 of SEQ ID NO: 1, in an allelic variant of SEQ ID NO: 1. The term “corresponding position” refers to a position in an allelic variant which is equivalent to a position defined with reference to SEQ ID NO: 1. Those skilled in the art will be able to determine a position in an allelic variant which corresponds to a position in SEQ ID NO: 1. Comparison of an allelic variant with the sequence set out in SEQ ID NO: 1, using for example the PILEUP program referred to below, will allow corresponding positions to be identified in an allelic variant, in particular positions in an allelic variant which correspond to position 104 in SEQ ID NO: 1.

Further preferred polymorphisms are ones which are in linkage disequilibrium with one or more of the above-mentioned polymorphisms.

In the method of the invention, the identity of one of the above-mentioned nucleotides may be determined for one or both alleles of the subject. Polymorphism at one or both of the alleles may be indicative of the dosage regime of the drug required for the treatment of the neurological condition.

A polymorphism which can be typed to determine the dosage regime of a drug used in the treatment of a neurological condition may be identified by a method comprising determining whether a candidate polymorphism in the SCN1A gene is: (i) associated with the response to the drug; or (ii) is in linkage disequilibrium with a polymorphism which is associated with the response to the drug, and thereby determining whether the polymorphism can be typed to determine the dosage regime of the drug.

A polymorphism to be typed according to the method of the invention may conveniently be detected by directly determining the presence of the polymorphic sequence in a SCN1A polynucleotide or protein of the subject. Such a polynucleotide is typically genomic DNA or mRNA, or a polynucleotide derived from these polynucleotides, such as a cDNA, which optionally may be in the form of a library. The detection method may be based on the detection of a difference in a characteristic between a SCN1A polynucleotide or a protein encoded by the SCN1A gene that carries the polymorphism and one which does not. For example, mobility of the proteins, such as mobility on a gel, may be detected. The polymorphism may be identified in a subject who has or is suspected of having epilepsy.

The polymorphism is typically detected by directly determining the presence of the polymorphism sequence in a SCN1A polynucleotide or protein of the individual. Such a polynucleotide is typically genomic DNA or mRNA, or a polynucleotide derived from these polynucleotides, such as in a library made using polynucleotide from the individual (e.g. a cDNA library).

The presence of the polymorphism may be determined in a method that comprises contacting a polynucleotide or protein of the subject with a specific binding agent for the polymorphism and determining whether the agent binds to a polymorphism in the polynucleotide or protein, the binding of the agent to the polymorphism indicating the dosage regime suitable for treating the subject.

Generally, the agent will also bind to flanking nucleotides and amino acids on one or both sides of the polymorphism, for example at least 2, 5, 10, 15 or more flanking nucleotide or amino acids in total or on each side. Generally, in the method, determination of the binding of the agent to the polymorphism can be done by determining the binding of the agent to the polynucleotide or protein. However, the agent may be able to bind the corresponding wild-type sequence by binding the nucleotides or amino acids which flank the polymorphism position, although the manner of binding will be different to the binding of a polynucleotide or protein containing the polymorphism, and this difference will generally be detectable in the method (for example this may occur in sequence specific PCR).

In the case where the presence of the polymorphism is being determined in a polynucleotide it may be detected in the double stranded form, but is typically detected in the single stranded form.

The agent may be a polynucleotide (single or double stranded) typically with a length of at least about 10 nucleotides, for example at least 15 or 20, up to about 25, 30, 35 or more nucleotides. The agent may be a molecule which is structurally related to polynucleotides that comprises units (such as purines or pyrimidines) able to participate in Watson-Crick base pairing. The agent may be a polypeptide, typically with a length of at least 10 amino acids, such as at least about 20, 30, 50 or more up to about 75, 100, 150, 200 or more amino acids. The agent may be an antibody, including a fragment such as of such an antibody which is capable of binding the polymorphism. Suitable fragments include Fv, F(ab′), F(ab′)2 and scFv fragments, as well as single chain antibodies. Furthermore, suitable antibodies and fragments thereof may be chimeric, CDR-grafted or humanised.

A polynucleotide agent which is used in the method will generally bind to the polymorphism and flanking sequence or wholly to the flanking sequence, of the polynucleotide of the individual in a sequence specific manner (e.g. hybridise in accordance with Watson-Crick base pairing) and thus typically has a sequence which is fully or partially complementary to the sequence of the polymorphism and/or flanking region. The partially complementary sequence shared sequence identity with the fully complementary sequence.

The agent may be a probe. This may be labelled or may be capable of being labelled indirectly. The detection of the label may be used to detect the presence of the probe on (and hence bound to) the polynucleotide or protein of the individual. The binding of the probe to the polynucleotide or protein may be used to immobilise either the probe or the polynucleotide or protein (and thus to separate it from a composition or solution).

The polynucleotide or protein of the individual may be immobilised on a solid support and then contacted with the probe. The presence of the probe immobilised to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The method may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

A probe may be used in a heteroduplex analysis based system to detect polynucleotide polymorphisms. In such a system when the probe is bound to polynucleotide sequence containing the polymorphism it forms a heteroduplex at the site where the polymorphism occurs (i.e. it does not form a double strand structure). Such a heteroduplex structure can be detected by the use of an enzyme which single or double strand specific. Typically the probe is an RNA probe and the enzyme used is RNAse H which cleaves the heteroduplex region, thus allowing the polymorphism to be detected by means of the detection of the cleavage products.

The method may be based on fluorescent chemical cleavage mismatch analysis which is described for example in PCR Methods and Applications 3, 268-71 (1994) and Proc. Natl. Acad. Sci. USA 85, 4397-4401 (1998).

A polynucleotide agent may be able to act as a primer for a PCR reaction only if it binds a polynucleotide containing the polymorphism (i.e. a sequence-specific or allele-specific PCR system). If such a polynucleotide agent is used, a PCR product will only be produced if the polymorphism is present in the polynucleotide of the individual. Thus, the presence of the polymorphism may be determined by the detection of the PCR product. Preferably the region of the primer which is complementary to the polymorphism is at or near the 3′ end of the primer. In one embodiment of this system the polynucleotide agent will bind to the wild-type sequence but will not act as a primer for a PCR reaction.

The method may be an RFLP based system. This can be used if the presence of the polymorphism in the polynucleotide creates or destroys a restriction site which is recognised by a restriction enzyme. Thus treatment of a polynucleotide comprising the polymorphism with a restriction endonuclease may lead to a different number of restriction products being produced as compared to the corresponding wild-type sequence. Thus the detection of the presence of particular restriction digest products can be used to determine the presence of the polymorphism.

The presence of the polymorphism may be determined based on the change which the presence of the polymorphism makes to the mobility of the polynucleotide or protein during gel electrophoresis. In the case of a polynucleotide, single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DDGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. Generally this assay uses an allele specific primer comprising the sequence around, and including, the polymorphism. The specific primer is labelled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridising to either allele 5′ of the polymorphism.

In the method of the invention, when the allele comprising the polymorphism is present Taq DNA polymerase adds nucleotides to the nonspecific primer until it reaches the specific primer. It then releases nucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye is not release from the quenching agent. Therefore by measuring the fluorescence emitted the presence or absence of the polymorphism can be determined.

In another method of detecting the polymorphism, a polynucleotide comprising the polymorphic region is sequenced across the region which contains the polymorphism to determine the presence of the polymorphism.

In general then, the detection of a polymorphism requires a polymorphism discrimination technique and a signal generation system. In addition, an optional amplification step is sometimes used.

Suitable polymorphism discrimination techniques, some of which may include PCR, and some of which have been described in detail above include:

General techniques, for example, DNA sequencing or sequencing by hybridisation;

Scanning techniques, for example protein truncation test (PTT) [not useful for the analysis of intron sequence polymorphism], single-strand conformation polymorphism analysis (SSCP), denaturing gradient gel eletrophoresis (DGGE), temperature gradient gel electrophoresis (TGGE), cleavase, heteroduplex analysis, chemical mismatch analysis (CMC) or enzymatic mismatch cleavage;

Hybridisation techniques, for example solid phase hybridisation (for example, dot blots, multiple allele specific diagnostic assay [MASDA], reverse dot blots, oligonucleotide arrays [DNA chips]) or solution phase hybridisation (for example, Taqman™ [U.S. Pat. No. 5,210,015 and U.S. Pat. No. 5,487,972], molecular beacons [Tyagi et al., Nature Biotechnology 14, 303, 1996 and WO-A-95/13399]);

Extension based techniques, for example amplification refractory mutation system (ARMS™), amplification refractory mutation system linear extension (ALEX™) [EP-B-332435] or competitive oligonucleotide priming system (COPS) [Gibbs et al., Nucleic Acids Research 17, 2347, 1989];

Incorporation based techniques, for example mini-sequencing or arrayed primer extension (APEX);

Restriction enzyme based techniques, for example restriction fragment length polymorphism (RFLP) or restriction site generating PCR;

Oligonucleotide based techniques: oligonucleotide ligation assay (OLA); and

Other types of assay, for example invader assay.

The above list provides examples of suitable polymorphism discrimination techniques and should not be construed as limiting.

Suitable signal generation or detection systems include:

Fluorescence based techniques, for example fluorescence resonance energy transfer, fluorescence quenching or fluorescence polarisation (GB-B-2228998); and

Other techniques, for example chemiluminescence, eletrochemiluminescence, raman, radioactivity, colorimetric, hybridisation protection assay, mass spectrometry.

This list of signal generation or detection systems is merely illustrative and should not be construed as limiting.

The oligonucleotides for use in amplifying a nucleic acid to be typed according to the invention may be any suitable oligonucleotides. The design of suitable oligonucleotides will be apparent to the person skilled in the art. Suitable oligonucleotides will be of any convenient length, for example up to about 50 nucleotides in length, up to 40 nucleotides in length, or more conveniently up to 30 nucleotides in length, such as for example, from about 8 to about 25 nucleotides in length or from about 12 to about 20 nucleotides in length. In addition, the oligonucleotides will may be designed such that the polymorphic site may be identified by the presence of a restriction endonuclease site. In this way, when and if the fragment is cut with an appropriate restriction endonuclease, the number of resulting fragments will be indicative of the nucleotide at the polymorphic site.

In general, suitable PCR primers will comprise sequences entirely complementary to the corresponding sequence to be amplified. However, if required, one or more, for example up to 3, up to 5 or up to 8 mismatches may be introduced, to introduce a convenient restriction enzyme site for example, provided that such mismatches do not unduly affect the ability of the primer to hybridize to its target sequence. Suitable primers may carry one or more labels to facilitate detection.

The sequences of two oligonucleotides which can be used in a method of the invention are those set out in SEQ ID NO: 5 (forward primer) and SEQ ID NO: 6 (reverse primer). A further combination of suitable oligonucleotides is set out in SEQ ID NO: 2 (forward primer) and SEQ ID NO: 4 (reverse primer).

It will be possible to use PCR primers which result in the amplification of the nucleotide at position 104 of SEQ ID NO: 1 and also at one or more, for example two, three, four or five, additional polymorphic positions within the SCN1A gene which may be associated with the response to the drug used in the treatment of a neurological condition. Therefore, it may be possible to determine the identity of more than one polymorphic site within the SCN1A gene simultaneously.

The presence of an A residue at position 104 of SEQ ID NO: 1 is indicative of a subject requiring a higher maintenance dose of the drug used in the treatment of a neurological condition than a subject having G at the said position. Where both alleles in a subject have an A residue at position 104 of SEQ ID NO: 1, the maintenance dose of the drug is even higher.

The invention also provides a test kit for use in a method for determining the dosage regime of a drug suitable for use in the treatment of a neurological condition in a subject. A test kit of the invention comprises means for typing the SCN1A gene of a subject.

Means for typing the SCN1A gene of a subject may comprise: means for determining the identity of a nucleotide at position 104 of SEQ ID NO: 1; means for determining the identity of a nucleotide at a corresponding position in an allelic variant of SEQ ID NO: 1; or means for determining the identity of a nucleotide in linkage disequilibrium with such a nucleotide.

The method of the invention may comprise typing the SCN1A gene and one or more additional genes in a subject. Typically, any such further gene will be associated with the response to the drug to be used to treat the subject, i.e. a method will comprise whether or not the subject has a polymorphism in one or more additional genes associated with the response to the drug suitable for use in the treatment of a neurological condition. That is to say, the SCN1A gene may be typed as part of a panel of variants that collectively allow determination of the dosage regime in the subject.

The method may also involve determining whether or not the subject has two or more polymorphisms in the one or more additional genes. Typically, at least one of the additional polymorphisms identified in the one or more additional genes is a causative factor in the response to a drug suitable for use in the treatment of a neurological condition or is in linkage disequilibrium with such a polymorphism.

Such a method may be carried out by typing the CYP2C9 gene. In this method, preferably the method if carried out to determine whether or not the subject carries the *3 allele (http://www.imm.ki.se/CYPalleles). The presence of one or two copies of the *3 allele is indicative of a subject requiring a lower maintenance dosage of the AED than a subject having fewer copies of the said allele.

In other words, in a preferred method of the invention the identity of nucleotides at a panel of polymorphic sites is determined. Preferably, at least one of these sites is the nucleotide at position 104 of SEQ ID NO: 1. Other polymorphic sites in a suitable panel may include further polymorphic sites, for example one, two, three, four or five further polymorphic sites, in the SCN1A gene which are, for example, associated with the response to a drug suitable for use in the treatment of a neurological condition.

Alternatively, or in addition, suitable polymorphic sites may be in one or more additional genes, for example one, two, three, four, five, ten, twenty, thirty, forty or fifty additional genes. Such genes may encode products which are associated with the response to the drug to be used in treatment of the subject. The drug may act directly on that the gene product and/or indirectly on the product. The drug may, for example, influence the way in which the gene product interacts with a further polypeptide, for example the polypeptide product of the SCN1A gene. The identity of a nucleotide at more than one, for example two, three, four or five, polymorphic sites within the one or more additional genes may be determined.

Thus, the method of the invention may be carried out by typing a panel of polymorphic sites. This may allow the dosage regime of a drug suitable for use in the treatment of a neurological condition to be further refined.

Any suitable means for determining the identity of one of the above-mentioned nucleotides may be included in a test kit of the invention. Typically, the means will comprise two oligonucleotides which can be used to amplify a polynucleotide from a subject which comprises the sequence set in SEQ ID NO: 1 or a fragment or allelic variant thereof. If the oligonucleotides may be used to amplify a fragment, that fragment will comprise at least one of the nucleotides set out above. Preferably, the oligonucleotides used will allow the nucleotides at all three of the positions identified above to be amplified. Suitable oligonucleotide pairs include: SEQ ID NO: 5 and SEQ ID NO: 6.

A test kit of the invention may optionally comprise, appropriate buffer(s), enzymes, for example a thermostable polymerase such as Taq polymerase and/or control polynucleotides. A kit of the invention may also comprise appropriate packaging and instructions for use in a method for determining the susceptibility of a subject to stroke. A test kit of the invention may also comprise a drug for use in the treatment of a neurological condition.

The invention allows the dosage regime of a drug for use in the treatment of a neurological condition to be determined in a subject.

Examples of drugs for which the dosage regime may be determined using the method of the invention include those whose action is mediated, either wholly or in part, by a sodium channel, for example a voltage-sensitive sodium channel, such as a sodium channel expressed in neurons. Preferably, the action of a drug for which the dosage regime may be determined using the method of the invention will be mediated by a voltage-sensitive sodium channel, type alpha. Thus, the action of the drug may be mediated by the product of one or more of the following genes: SCN1A, SCN2A1, SCN2A2, SCN3A, SCN4A, SCN5A, SCN6A, SCN8A, SCN9A, SCN10A, SCN11A or SCN12A. The action of the drug is most preferably mediated by the product of the SCN1A gene.

Such mediation may be direct, that is to say the drug may be one which acts either wholly or partly on such a sodium channel. Alternatively, the mediation may be indirect. For example, the drug may be one which acts on a polypeptide which then interacts with or binds to a suitable sodium channel. If the action of the drug is mediated by a voltage-sensitive sodium channel, type alpha, (for example by the product of the SCN1A gene, the drug may preferably act by binding to the alpha subunit.

Preferred drug for which the dosage regime may be determined using the method of the invention include anti-epileptic drugs (AEDs). Suitable anti-epileptic drugs will preferably be ones which are capable of enhancing GABAergic pathways, inhibiting excitory glutaminergic pathways , inhibiting excess neuronal firing or inhibiting seizure spread. A suitable AED may be an anti-convulsant drug.

Such drugs include carbamazepine, phenytoin, phenytoin sodim, lamotrigine, topimiramate, oxcarbamazepine and valproate or a derivative or analog of any one thereof. A drug for which the dosage regime may be determined according to the method of the invention may be derivative or an analog of one of such a drug. Also, the drug may be formulated as a modified-release formulation, for example carbamazepine may be in the form of a modified-release formulation (CBZ-MR).

Carbamazepine is also know as 5H-dibenz[b,f]azepine-5-carboxamide. The preparation of carbamazepine is described in U.S. Pat. No. 2,948,718. The method of the invention may be used to determine the dosage regime of a derivative or analog of carbamazepine.

For example, suitable derivatives or analogs may be substituted or unsubstituted at the 10- or 11-position. The 10- or 11-position may be substituted with mono- or divalent substituents selected from oxa, halogen or hydroxy groups, preferably oxa- or hydroxy-groups. Where there is an oxa- or a hydroxy-group at the 10-position, the 11-position is preferably unsubstituted and vice versa. Preferred compounds of this sort include 10-oxacarbazepine and 10-hydroxy-10,11-tetrahydrocarbamazepine. The latter compound has a chiral centre and may be used as its racemic mixture.

More particularly, carbamazepine derivatives for which the method of the invention can be used to determined the dosage regime include those of formula (I) as set out in WO 2004/014391 (PCT/EP2003/008669), wherein (a) R₁ represents hydrogen, and R₂ represents hydroxy or C₁-C₃ alkyl carbonyloxy, or (b) R₁, and R₂ together represent an oxo group, and their pharmaceutically acceptable salts.

The preparation of the compound of formula (I) wherein R₁ is hydrogen and R₂ represents hydroxy and of its pharmaceutical acceptable salts is described, for example in U.S. Pat. No. 3,637,661. This compound, monohydroxycarbamazepine (10-hydroxy-10,11-dihydro-carbamazepine), the main metabolite of the antiepileptic oxcarbazepine is well known in the art (see for example Schuetz et al., Xenobiotica (GB) 16 (8), 769-778 (1986)).

The preparation of the compound of formula (I) wherein R₁ is hydrogen and R₂ represents C₁-C₃ alkyl carbonyloxy and of the pharmaceutically acceptable salts thereof is described, for example in U.S. Pat. No. 5,753,646.

The carbamazepine derivatives of formula (I) wherein R₁ represents hydrogen, and R₂ represents hydroxy or C₁-C₃ alkyl carbonyloxy constitute chiral compounds. For the purposes of the present invention, the chiral compounds disclosed herein can be employed in the form of racemates, in mixtures comprising one enantiomer in excess (e. g. more S-10-hydroxy-10,11-dihydro-carbamazepine than R-10-hydroxy-10,11-dihydro-carbamazepine) or in enantiomerically pure form (e. g. pure S-10-hydroxy-10,11-dihydro-carbamazepine or pure R-10-acetoxy-10,11-dihydro-carbamazepine).

The compound of formula (I) wherein R₁ and R₂ together represent an oxo group is known as oxcarbazepine(10-oxo-10,11-dihydro-5H-dibenz[b, f]azepine-5-carboxamide.

The method of the invention may also be used in determining the dosage regime of the dibenzazepine derivatives described in U.S. Pat. No. 3,221,011. These compounds have the general structure set out in U.S. Pat. No. 3,221,011 where Am may signify the radical —NH2, a monomethyl, monoethyl, mono-n-propyl, mono-isopropyl, mono-n-butyl, mono-isobutyl or mono-tert-butylamino radical, a dimethyl, diethyl, di-n-propyl, diisopropyl, di-n-butyl, diisobutylamino radical, a methylethylamino, methyl-n-propylamino, methylisopropylamino or ethylisopropylamino radical.

The method of the present invention is applicable to all of the carboxamides described above. The term “carboxamides” as used herein includes, but is not limited to oxcarbazepine, 10-hydroxy-10,11-dihydrocarbamazepine and 10-acetoxy-10,11-dihydrocarbamazepine.

Phenytoin (5,5-diphenylhydantoin), phenytoin sodium and procedures for their manufacture are well know, for example from U.S. Pat. No. 4,696,814, U.S. Pat. No. 4,642,316 and U.S. Pat. No. 2,409,754. The method of the invention may be used to determine the dosage regime of a derivative or analog of phenytoin.

For example, U.S. Pat. No. 3,595,862 describes N,N′-bis(acyloxymethyl)5,5-diphenylhydantoin compounds. These compounds include those in which the acyloxy groups are acetoxy, acryloxy, methacryloyloxy, propionoxy or benzoyloxy.

Further derivatives of phenytoin include the 5,5-diphenylhydantoins or salts thereof disclosed in U.S. Pat. No. 4,260,769. The method of the invention may be used to determine the dosage regime of such phenytoin derivatives. These phenytoin derivatives have the formula set out in U.S. Pat. No. 4,260,796, or the pharmaceutically acceptable acid addition or basic salt, C1 to C4 alkylhalide quaternary salt or N-oxide thereof, wherein R represents H or —CH(R₁)—O—P—(O)(OH)₂ wherein R₁ is selected from the group consisting of H, C₁-C₇ straight or branched chain alkyl, with the proviso that both the R's cannot simultaneously be hydrogen.

Preferred phenytoin derivatives of the type described above include:

3-Ethoxycarbonyloxymethyl-diphenylhydantoin;

3-Benzyloxycarbonyloxymethyl-diphenylhydantoin;

3-(2′,2′,2′-Trichloroethyloxycarbonyloxymethyl)-diphenylhydantoin;

3-(N,N-Dimethylglycyloxymethyl)-diphenylhydantoin;

3-(1-Piperidylacetyloxymethyl)-diphenylhydantoin;

3-Benzoyloxymethyl-diphenylhydantoin;

3-p-Toluyloxymethyl-diphenylhydantoin;

3-Picolinoyloxymethyl-diphenylhydantoin; 9.3-Nicotinoyloxymethyl-diphenylhydantoin;

3-N-Formylglycyloxymethyl-diphenylhydantoin;

3-Glycyloxymethyl-diphenylhydantoin;

3-N-Benzyloxycarbonylglycyloxymethyl-diphenylhydantoin;

3-Methylsuccinyloxymethyl-diphenylhydantoin;

3-(N,N-Dimethylsuccinamyloxymethyl)-diphenylhydantoin;

3-(N,N-Diethylsuccinamyloxymethyl)-diphenylhydantoin;

3-(N,N-Trimethylglycyloxymethyl)-diphenylhydantoin;

3-(N,N,N-Triethylglycyloxymethyl)-diphenylhydantoin;

3-[α-(N,N-Dimethylglycyloxy)ethyl]-diphenylhydantoin;

3-[α-(1-Piperidylacetyloxy)ethyl]-diphenylhydantoin;

3-(α-Benzoyloxyethyl)-diphenylhydantoin;

3-(α-Picolinoyloxyethyl)-diphenylhydantoin;

3-[α-(N-Formylglycyloxy)ethyl]-diphenylhydantoin;

3-[α-(N-Benzyloxycarbonylglycyloxy)ethyl]-diphenylhydantoin;

3-(α-Methylsuccinyloxyethyl)-diphenylhydantoin;

3-[α-(N,N-Dimethylsuccinamyloxy)ethyl]-diphenylhydantoin;

3-[α-(N,N,N-Trimethylglylcyloxy)ethyl]-diphenylhydantoin chloride;

3-(α-Ethoxycarbonyloxybenzyl)-diphenylhydantoin;

3-[α-(N,N-Dimethylglycyloxy)benzyl]-diphenylhydantoin;

3-[α-(1-Piperidylacetyloxy)benzyl]-diphenylhydantoin;

3-(αPicolinoyloxybenzyl)-diphenylhydantoin;

3-[α-(N-Formylglycycloxy)benzyl]-diphenylhydantoin;

3-[α-(N-Benzyloxycarbonylglycyloxy)benzyl]-diphenylhydantoin;

3-(α-Methylsuccinyloxybenzyl)-diphenylhydantoin;

3-[α-(N,N-Dimethylsuccinamyloxy)benzyl]-diphenylhydantoin;

3-[α-(N,N,N-Trimethylglycyloxy)benzyl]-diphenylhydantoin chloride;

3-(N,N-Dimethylglycylthiomethyl)-diphenylhydantoin;

3-(1-Piperidylacetylthiomethyl)-diphenylhydantoin;

3-p-Toluylthiomethyl-diphenylhydantoin;

3-Picolinoylthiomethyl-diphenylhydantoin;

3-Nicotinoylthiomethyl-diphenylhydantoin;

3-N-Formylglycylthiomethyl-diphenylhydantoin;

3-Glcylthiomethyl-diphenylhydantoin;

3-(N,N-Diethylsuccinamylthiomethyl)-diphenylhydantoin;

3-(N,N,N-Trimethylglycylthiomethyl)-diphenylhydantoin chloride;

3-(N,N,N-Triethylglycylthiomethyl)-diphenylhydantoin chloride;

3-Phosphoryloxymethyl-diphenylhydantoin;

3-Succinyloxymethyl-diphenylhydantoin; and

3-Glutaryloxymethyl-diphenylhydantoin.

Stable pharmaceutical compositions of 3-(hydroxymethyl)-5,5-diphenylhydantoin disodium phosphate ester are disclosed in U.S. Pat. No. 4,925,860 and these represent further phenytoin derivatives for which the method of the invention may be used to determined the dosage regime. Specific examples of such phenytoin derivatives include 3-(hydroxy-methyl)-5,5-diphenylhydantoin phosphate ester disodium salt, a prodrug of phenytoin and also 3-(hydroxy-methyl)-5,5-diphenylhydantoin disodium phosphate ester. This compound is also variously known as fosphenytoin sodium; the disodium salt of prophenytoin; 5,5-diphenyl-3-[(phosphonooxy)methyl]-2,4-imidazolidinedione disodium salt; 3-phosphoryloxymethyl-5,5-diphenylhydantoin disodium salt; or 3-(hydroxymethyl)-5,5-diphenylhydantoin disodium phosphate ester.

Combination therapy may be carried out, in which case the method of the invention may be used to determine the dosage regime for a combination of drugs suitable for use in the treatment of a neurological condition. Generally, however, combination therapy will be used only when attempted monotherapy with several alternative drugs has proven ineffective. Combination therapy may increase toxicity and drug interactions may occur.

Any one of more, for example, two, three or four of the drugs mentioned above may be incorporated in a kit of the invention and in products of the invention.

The neurological condition for which the dosage regime may be determined according to the method of the invention is typically one which may be treated using a drug which acts, either wholly or in part, on a sodium channel, for example a voltage-sensitive sodium channel, such as a sodium channel expressed in neurons. Preferably, the neurological condition is one which is treated using a drug which acts on a voltage-sensitive sodium channel, type alpha, for example by acting on one or more of: SCN1A, SCN2A1, SCN2A2, SCN3A, SCN4A, SCN5A, SCN6A, SCN8A, SCN9A, SCN10A, SCN11A or SCN12A. Preferably, the neurological condition is one which acts on the product of the SCN1A gene. This is intended to encompass neurological conditions which may be treated using drugs which act either directly on one of more of the gene products set out above (for example the SCN1A gene product), for example by binding to an alpha subunit, or indirectly, for example via interaction with a polypeptide which then itself interacts with the said gene product.

Specific examples of neurological conditions for which the dosage regime may be determined using the method of the invention include headache, chronic head and body pain syndromes, trigeminal neuralgia, manic-depressive psychosis, a mood disorder (i.e. the dosage regime for mood stabilisation may be determined) and depression.

The method is particularly applicable to the determination of the dosage regime of a drug used in the treatment of epilepsy. Epilepsy is heterogeneous and the term encompasses a number of conditions. Broadly, there are two types of epilepsy: focal/partial; and generalised. Carabamazepine is generally used to treat only the former and therefore the method of the invention is particular suitable for determining the dosage regime for a subject who will be treated for focal epilepsy, with or without secondary generalisation, using cabamazepine.

Once the dosage regime of a drug for treating a neurological condition in a subject has been determined, the subject may be treated with that drug according to that treatment regime.

In the treatment of a neurological condition, for example epilepsy, an effective (maintenance) dosage of a drug, for example an AED, may be administered to a subject at regular intervals. The effective dosage is generally not, however, the dosage that is initially administered to the subject. Typically, a dosage lower than that likely ultimately to be effective is administered when treatment is initiated. If symptoms, for example seizures, continue, the dosage is progressively increased until the effective dosage is reached. If on the other hand, the starting dosage is too great and side effects result, for example dizziness, the dosage may be progressively decreased until the effective dosage is arrived at. In effect therefore, the dosage is titrated up until either symptoms such as seizures stop, or the subject develops side-effects.

In the context of this invention, “dosage regime” indicates the schedule of treatment given to a subject in establishing an effective/maintenance dosage of a drug suitable for use in the treatment of a neurological condition. This encompasses the amount of a drug given, as starting dosage, and the rate at which the dosage is then increased towards the maintenance dosage.

The rate at which the dosage may be increased towards the maintenance dosage covers both the increase in dosage at each interval and the time interval at which the increased dosages are administered. The term “dosage regime” also covers the predicted maximum dosage tolerated.

The genotype of the subject as determined in accordance with the invention will be indicative of the likely maintenance dosage required by the subject. Accordingly, the dosage regime may be tailored to the subject so that a maintenance dosage may be achieved more rapidly.

For example, maximum dosage of an AED tolerated by a subject decreases in AA, AG and GG individuals respectively. Using this information, a suitable dosage regime may be devised so that the appropriate maintenance dosage may be achieved more quickly than would be the case using a solely titration-based approach (i.e. increasing dosage gradually over a period of time until symptoms are alleviated or side-effects develop). That is to say, the appropriate dosage may be built up more quickly than would be the case in a subject where there is no indication as to the maintenance dosage likely to be tolerated by that subject (other than the population average).

Thus, in a typical dosage regime, the dosage may be increased at intervals of no less than 2 weeks. The determination of the genotype of the subject may indicate that the subject will tolerate a higher or lower than average maximum dosage of the drug. A physician or veterinary surgeon may be advised accordingly, so that the indicated maximum dosage is not exceeded.

Thus, in the treatment of epilepsy, for example, a typical dosage regime involves initial treatment by administration of 200 mg/day of an AED such as CBZ or CBZ-MR. Typically, this dosage is increased by 200 mg increments at intervals of no less than 2 weeks until the maintenance dosage is reached.

However, it may be possible to increase the dosage more rapidly in a subject once the SCN1A gene has been typed. That is because the maximum dosage of an AED tolerated by that individual will be known and this may be taken into consideration when treating the individual.

In particular, a subject which is heterozygous or homozygous for the rs3812718 polymorphism is likely to require a higher maintenance dosage of an AED than a subject which does not carry the polymorphism. An individual which does not carry the polymorphism is likely to require a lower maintenance dosage. With this in mind, a dosage regime may be used in which the dosage is increased at intervals of less than one week, for example every ten days, every seven days, every five days or every three days. In addition, the starting dosage may be greater than 200 mg/day, for example 250 mg/day, 300 mg/day, 350 mg/day, 400 mg/day, 450 mg/day or 500 mg/day. Also, the increment by which the maintenance dosage is increased may itself be increased. Thus, the dosage may be increased at increments of 250 mg, 300 mg, 350 mg, 400 mg, 450 mg or 500 mg.

In view of the genotype of the subject, the likely dosage which should not be exceeded will be known. As this dosage is approached, the dosage regime can be tailored so that the dosage increase interval is increased and/or the dosage increment is decreased.

A subject which is homozygous for the rs3812718 polymorphism (AA of position 104 of SEQ ID NO: 1) is likely to require a higher maintenance dosage of an AED than a subject which does not carry the polymorphism. Thus, the starting dosage may be increased, the dosage increase interval decreased and the dosage increment increased to a greater degree than would be the case for a subject heterozygous for the polymorphism (AG) or for a subject which does not carry the polymorphism.

Critically, however, knowledge of the genotype of the subject should allow a dosage regime to be determined such that the maintenance dosage is achieved more quickly than would be the case than in a subject where the genotype is not known.

The starting dosage may be increased, the dosage increase interval decreased and the dosage increment increased typically to a smaller degree in a subject which does not carry the rs3812718 polymorphism (as compared to a subject which is either heterozygous or homozygous for the polymorphism). However, in view of the fact that the likely maximum dosage will be known, it will still be possible to achieve the maintenance dosage more quickly would be the case for a dosage regime based simply on titration against patient response.

Any combination of the starting dosage, the dosage increase interval and the dosage increment may be used so as to arrive at a dosage regime of the invention.

The initial dose of the drug used in the treatment or prophylaxis of a neurological condition may be determined according to various parameters, especially according to the substance used; the age, weight and condition of the patient to be treated; the route of administration; and the dosage regime as determined using the method of the invention. Again, a physician will be able to determine the required route of administration and dosage for any particular patient.

Drugs which are used in the treatment of a neurological condition may also be used in the manufacture of a medicament for use in a method of treatment or prophylaxis of that neurological condition in a subject using the dosage regime determined according to the method of the invention. Thus, the condition of a subject may be improved by treatment in accordance with the dosage regime determined using a method of the invention. A therapeutically effective amount of a drug for use in the treatment of a neurological condition may be given to a subject in accordance with a dosage regime determined using a method of the invention.

A drug which is used according to a dosage regime determined using the method of the invention may be administered in a variety of dosage forms. Thus, they may be administered orally, for example as tablets, troches, lozenges, aqueous or oily suspensions, dispersible powders or granules. The drug may also be administered parenterally, either subcutaneously, intravenously, intramuscularly, intrasternally, transdermally or by infusion techniques. The drug may also be administered as suppositories. A physician will be able to determine the required route of administration for each particular patient.

The formulation of the drug will depend upon factors such as the nature of the drug, whether a pharmaceutical or veterinary use is intended, etc. A drug which is to be used to treat a neurological condition may be formulated for simultaneous, separate or sequential use.

Products containing means for typing the SCN1A gene of a subject and a drug fur use in the treatment of a neurological condition as a combined preparation for simultaneous, separate or sequential use in a method of treatment of the human or animal body by therapy are provided by the invention. Thus, products of the invention may comprise both means for the determination of a dosage regime of a drug and means for therapy (the drug itself).

An drug suitable for use in the treatment of a neurological condition in a dosage regime determined by the method of the invention is typically formulated for administration with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

The following Example illustrates the invention:

### EXAMPLE

### Materials and Methods

In this study we have considered the known functional alleles *2 and *3 at the CYP2C9 gene, and the putatively functional 3435C>T polymorphism in the ABCB1 gene. As no common functional variants are known for SCN1A, we used a haplotype tagging strategy (Weale et al., Am J. Hum. Genet. 73, 551-565, 2003). We have related variation in all three genes to the maximum dose of phenytoin in 281 patients treated with phenytoin. For carbamazepine, we related variation in both SCN1A and ABCB1 to maximum dose in 425 patients. Finally, we tested for association with presence or absence of ADRs. There were no significant violations of Hardy Weinberg equilibrium after Bonferroni corrections for multiple comparisons. In most, but not all cases the maximum dose used here will also be the maintenance dose, since starting doses tend to be lower than what is required (see discussion below).

Subjects

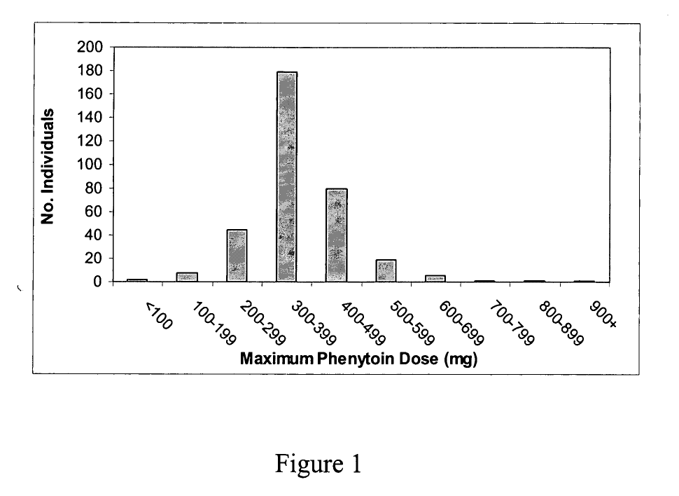

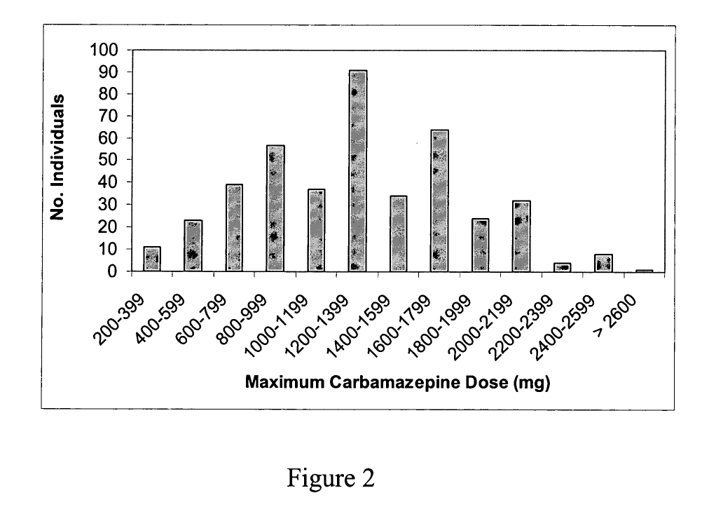

The study was approved by the relevant institutional Ethics Committees. Patients who self-identified, or were determined by the treating physician, as being of North Western European origin with a diagnosis of epilepsy were recruited from the specialised epilepsy clinic of the National Hospital for Neurology and Neurosurgery after written informed consent was obtained. Extensive clinical data were obtained and stored in a computerized database. The DNA collection contains DNA from patients over the last three years and a number of patients who are no longer attending clinic. We identified 448 patients who were treated with phenytoin, of whom 119 were continuing treatment at the time of recruitment and the remainder had stopped treatment. DNA and dose information was available for 281 patients (FIG. 1). We identified 533 patients who were treated with carbamazepine; DNA and dose information was available for 425 (FIG. 2). Of the 425 patients exposed to carbamazepine, 240 were not included in the phenytoin analyses.

The following clinical details were recorded whenever available: date when phenytoin/carbamazepine was started and stopped, maximum dosage reached, response and occurrence of ADRs. Although more details are available in the relevant clinical notes, it has not been possible in all cases to review these notes.

Brain tissue

Thirty-five pairs of surgically resected peri-lesional temporal neocortex and hippocampus brain tissue were selected at random from the archives of frozen tissue at the National Hospital for Neurology and Neurosurgery. In each case therapeutic surgery had been undertaken to relieve chronic drug resistant epilepsy. All tissue had been flash-frozen in liquid nitrogen within 30 minutes of resection, and stored at 80° C. until use. Routine detailed histological examination of the fixed (unaffected) temporal lobe and hippocampus from each case had shown hippocampal sclerosis and the absence of epileptogenic pathology in the temporal lobe. In each case, written informed consent had been obtained from the patient for the use of resection material for research approved by the institutional ethics committee; all samples were irreversibly anonymised prior to analyses. Twenty-three brain samples from patients with Parkinson's Disease (PD) were obtained from the Brain Bank at the Institute of Neurology. Samples were anonymised, and ethical permission for this study was obtained from the joint research ethics committee of the National Hospital for Neurology and Neurosurgery and Institute of Neurology.

Genotyping

CYP2C9*2 and CYP2C9*3 genotyping was performed using pre-developed Taqman assay reagents for allelic discrimination (Applied Biosystems) according to the manufacturers instructions.

The four SCN1A tSNPs (rs590478, rs8191987, rs3812718 and rs2126152) had been previously genotyped using Taqman assays (Depondt and colleagues, in preparation). (These tSNPs correspond to SNP1, SNP5, SNP7 and SNP8 in (Weale et al., 2003, supra) and have frequencies of 0.24, 0.13, 0.45 and 0.32 in 384 unrelated control individuals from the British twin registry (Andrew et al., Twin Res. 4, 464-477, 2001)). The authors found the proportion of haplotype diversity explained by these tSNPs to be 94% (criterion 2 in TagIT), and the average haplotype r² (criterion 5 in TagIT (Weale et al. TagIT version 2.02 available at http://popgen.biol.ucl.ac.uk/software.html. 2004. Ref Type: Computer Program)) to be 0.8 (using data from Weale et al., 2003, supra). These values also mean that common SNPs (minor allele frequency >8%) in the gene are generally predicted well by the subset of tSNPs, and that little loss of power is therefore expected in typing the tSNPs instead of typing directly a causal SNP, assuming its minor allele frequency is sufficiently high.

ABCB1 3435C>T genotypes had been previously determined using direct sequencing (Soranzo et al., Genome Res. 14, 1333-1344, 2004).

Nucleic Acid Purification From Brain Samples and RT-PCR

Genomic DNA was extracted from approximately 25 mg of brain tissue using a Wizard Genomic DNA Purification Kit (Promega) according to the manufacturer's conditions. Total RNA was isolated from approximately 30 mg of the same brain tissue using the Lipid Tissue Purification Kit (Qiagen), according to manufacturer's conditions. The RNA was quantified spectrophotometrically at 260 nm, and 1 μg RNA of each sample was reverse-transcribed to cDNA using the High Capacity cDNA Synthesis Kit (Applied Biosystems), in standard conditions. RT-PCR was carried out on a volume of cDNA corresponding to 10 ng starting RNA.

RT-PCR was used to determine that exon SN is present in human mRNAs using commercially obtained human foetal total brain mRNA (Stratagene). Adult human cDNA was obtained from brain tissues and amplified with primers flanking exon 5 designed to amplify exons 5A and 5N equally:

| | |
|---|---|
| F-CCACCTCTGCCCTGTACATT | (SEQ ID NO:5) |
| R-CTCCCACAATGGTTTTCAGG | (SEQ ID NO:6) |

The resulting fragment was digested with AvaII, which cuts only copies containing exon 5N, for >2 hours and separated on 3% agarose gels in the presence of ethidium bromide. The relative intensity of the 5N product and 5A product was measured for each sample using the Syngene package. The ratio of 5A to 5N was corrected for the molecular weight of the two products.

Statistical Analyses

All regression analyses were implemented in the usual way using STATISTICA (StatSoft Inc, Tulsa, USA) with phenytoin/carbamazepine dose as the dependent variable and genotype score(s) as the independent predictor(s).

Multi-locus association analysis was carried out using the score test (Schaid et al., Am. J. Hum. Genet. 70, 425-434, 2002). Briefly, the method uses an expectation maximisation (EM) algorithm to infer haplotypes from a set of unrelated individuals. A score statistic measuring the association of the inferred haplotypes with the quantitative trait is then estimated. All these analyses were implemented in the software package R (version 1.9.1 (R Development Core Team—R: A language and environment for statistical computing. Wien, Austria: R Foundation for Statistical Computing, 2004. 2002.)).

Standard χ² analyses were used to compare genotype frequencies between individuals with and without ADRs.

A Student's t-Test was used to compare ratios of SCN1A with 5A to SCN1A with 5N by SCN1A IVS5-91 G>A genotype.

### Results

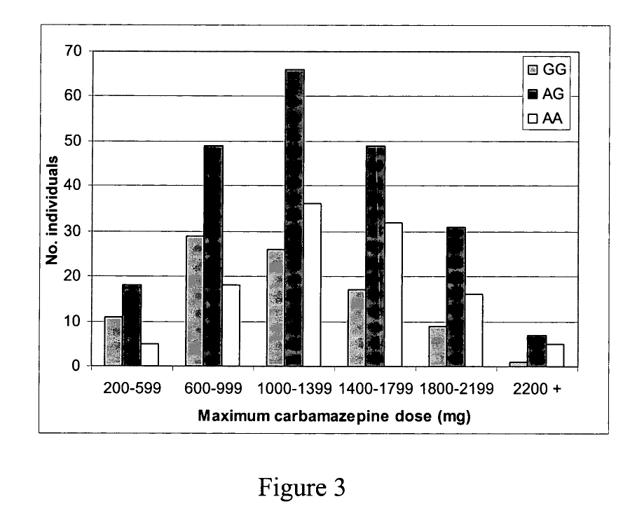

For carbamazepine we find that one of the SCN1A tagging SNPs (SNP7, IVS5-91 G>A, or rs3812718) is highly associated with maximum dose. A regression model allowing arbitrary effects for each genotype is significant at the level P=0.0051 (uncorrected). The genotypic effects however are consistent with additive effects, and under a regression model restricted to additive effects the significance is P=0.0014 (uncorrected). These results remain significant after Bonferroni correction for five tests (four in the SCN1A gene, and one in the ABCB1 gene). Maximum doses averaged 1313 mg, 1225 mg and 1083 mg for AA, AG and GG individuals (with genotype counts of 112, 220 and 93 individuals respectively, FIG. 3). A weighted linear haplotype regression, using all the tSNPs, does not increase significance.

For phenytoin we find that the CYP2C9*3 allele shows significant association with maximum dose, with P=0.0066 (uncorrected). As there was only one individual homozygous for the *3 allele in our cohort we excluded this genotype from our regression model (though this single observation follows the same trend of reduction in maximum dose). This value remains significant after Bonferroni correction for 7 independent genotyping tests (four tests in the SCN1A gene, two in CYP2C9, and one in ABCB1). Mean phenytoin doses for individuals with 0, 1 or 2 copies of the *3 allele were 354 mg, 309 mg and 250 mg, respectively (but notice numbers for these three genotypes are 229, 39, 1 respectively). CYP2C9*2 did not show a significant association with dose. A multiple regression analysis on combined *2 and *3 genotype did not support a significant role for *2.

The SCN1A IVS5-91 G>A polymorphism is also associated with the dosing of phenytoin with P=0.014 (uncorrected) under an unrestricted regression model and P=0.0045 (uncorrected) under an additive model. The latter model would appear to be indicated from the apparent additive effect of the SCN1A genotype on carbamazepine dosing explained above. Under the additive model significance is retained after correcting for 7 independent tests. The unrestricted model shows only a trend after correction. Maximum phenytoin doses averaged 373 mg, 340 mg and 326 mg for AA, AG and GG individuals, respectively (genotype counts of 73, 109 and 60, respectively). A weighted linear haplotype regression, using all the tSNPs, does not increase significance. When the combined CYP2C9*3 and SCN1A IVS5-91 G>A are considered, the doses range from a mean of 250 mg for the single *3*3/GG individual to 297 mg for (Schaid et al., 2002, supra)*1*3/AG individuals and 377 mg for (the 62)*1*1/AA individuals, P=0.014, uncorrected under unrestricted model. (The single *3*3/GG individual has again been excluded however it followed the same trend).

Of the patients included in the carbamazepine analysis, 185 also had been included in the phenytoin analysis. When these patients are not included, the result remains significant under an additive model with P=0.0063 (uncorrected). An unrestricted model gives P=0.020 (uncorrected). The phenytoin and carbamazepine results therefore provide a functional replication of the effect of the SCN1A variant.

The ABCB1 3435C>T polymorphism shows no association with dosing for either phenytoin or carbamazepine.

We report here for the first time that the IVS5-91 G>A polymorphism in SCN1A affects the alternative splicing of exon 5 (FIG. 4). This polymorphism is located in the 5′ splice donor site of a highly conserved alternatively spliced exon apparently expressed mainly in foetuses (5N) (Copley, Trends Genet. 20, 171-176, 2004). The major allele (A) disrupts the consensus sequence of the foetal exon (5N) possibly reducing the expression of this exon relative to the adult exon (5A). A similar splicing event occurs in the German cockroach sodium channel gene, para^{CSMA}, but in a different domain (domain III). Substituting aspartic acid into the S3-S4 linker is associated with altered voltage-gating and sensitivity to the insecticide deltamethrin (although there are other substitutions between exons) (Tan et al., J. Neurosci. 22, 5300-5309, 2002).

We first studied human foetal whole brain mRNA and confirmed that exon 5N is present (data not shown). Next we amplified the region including exon 5, in adult human cDNA samples derived from brain tissue, with primers flanking that exon. The product of this PCR was digested with the restriction enzyme Ava II, which cuts only in copies containing exon 5N. In the mRNA purified from foetal brain (the genotype is unavailable from the commercial mRNA) more than 60% of the amplified SCN1A mRNA contained exon 5N. We also looked in adult brains derived from a Parkinson's disease brain bank. In these adult brains without a history of epilepsy the levels of SCN1A with exon 5N were much lower with 9.5±0.7% (n=23) over all genotypes with individuals with the AA genotype having slightly, but not significantly less 5N (8.6±0.75%, n=5) than individuals of either AG (9.94±1.09% n=14) or GG (9.2±0.96% n=4).

There is evidence that seizures can upregulate the inclusion of exon 5N in neuronal sodium channels in rodents (Gastaldi et al., Brain Res. Mol. Brain Res. 44, 179-190, 1997). We therefore also assessed the percentage of SCN1A mRNA containing exons 5A and 5N in brain resection tissue derived from patients undergoing surgery for refractory epilepsy. In these tissues the amount of SCN1A containing exon 5N was significantly upregulated in the temporal lobe (TL) relative to the hippocampus (Hipp) in individuals with the permissive GG genotype (TL 14.6±1.2%, Hipp 11.2±0.8% n=8), P=0.023, but not in individuals with the AA (TL 11.0±1.5% Hipp 11.7±2.2% n=10) or AG (TL 15.1±1.3% Hipp 13.4±1.9 n=8) genotypes (FIG. 4, bottom).

Taken together, these results show that seizures influence the proportions of alternative transcripts of the SCN1A gene. We also show that the influence of seizures is dependent on genotype with the GG permissive genotype resulting in a significant increase of the 5N form in the temporal lobe relative to the hippocampus. These results do not make clear how the IVS5-91 G>A splice site polymorphism influences sensitivity to carbamazepine and phenytoin. Future work focused on precise expression patterns in subregions of the hippocampus and functional assays of drug sensitivity of the channels encoded by 5A and 5N may eventually help to clarify this. Finally, it is also possible that the presence/absence of 5N during development leads to changes in other sodium channels, which change adult sensitivity to sodium channel blockade. This work however demonstrates a novel phenomenon in humans of how seizures influence neuronal function as neither rats nor mice possess a functional copy of exon 5N in SCN1A (Schorge, S., unpublished observation).

Given the demonstrated association of the IVS5-91 G>A polymorphism and 5N and 5A levels, and its presence in a splice donor consensus site, it is very likely that this polymorphism is itself the causal polymorphism for altered sensitivity to phenytoin and carbamazepine. Because of the extensive LD throughout the gene it is formally possible that the causal variant lies elsewhere (Weale et al., 2003, supra). We have however, undertaken exhaustive screening of the SCN1A exons and intron-exon boundaries and have not found any other common variants that are predicted to have functional effects (Depondt and colleagues, in preparation).

No associations were found between any genotype and presence of an ADR or presence of the subset of ADRs that are central nervous system-related.

Stratification Does Not Explain the Association:

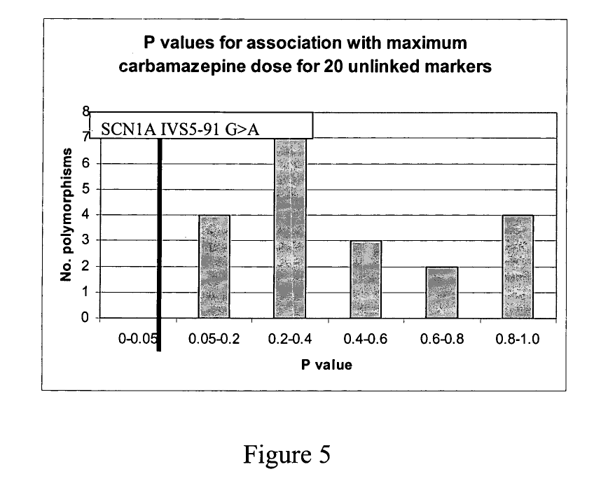

Although all patients in this study self-identified, or were determined by the treating physician, as being of North Western European origin, cryptic stratification can still drive spurious associations between polymorphisms and phenotypes, including drug responses (Freedman et al., Nat. Genet. 36, 388-393, 2004). One approach for checking whether stratification is present for a given phenotype, termed genome control, is to assess the association of that phenotype with markers from elsewhere in the genome that are not linked to the associated polymorphism under study (Reich et al., Genet. Epidemiol. 20, 4-16, 2001). As part of other projects in the laboratory not related to this work on dosing requirements for carbamazepine and phenytoin we have typed a series of unlinked polymorphisms. We have assessed the degree of association of each of these polymorphisms with carbamazepine dosing. We find none of these polymorphisms is significantly associated with maximum dose (FIG. 5). We may therefore set a threshold on the probability that the significant association for the SCN1A polymorphism is influenced only by stratification. Under this null hypothesis the distribution is as described by our set of genome control markers, and the SCN1A polymorphism is a significant outlier in this distribution with p<0.05. As noted in Reich et al., 2001 (supra), this formulation of genomic control is conservative.

We also note that some of the polymorphisms considered were chosen for relevance to epilepsy and so could possibly influence dose requirements, but in the context of using them for genome control this is a conservative effect. This analysis therefore effectively rules out stratification as an explanation of our results.

### Discussion

We have identified functional polymorphisms that are highly associated with the dose used in regular clinical practice for two leading AEDs, phenytoin and carbamazepine. For phenytoin, a well-known low-activity variant in the CYP2C9 gene associates with dose, as does a newly described functional variant in the SCN1A gene, encoding the target of phenytoin. The SCN1A variant is also highly associated with dosing of carbamazepine, thus providing functional replication of the effect of this variant. This SCN1A variant is the first polymorphism in a drug target associated with the use of an AED, and one of only a handful of target polymorphisms for which there is strong evidence of an effect on clinical drug use (Goldstein et al., Nat. Rev. Genet. 4, 937-947, 2003). Furthermore this is the first demonstration of pharmacologic significance of alternative splicing in a human sodium channel. Although there are other examples of alternative splicing in other human sodium channel genes, none has been associated with functional effects (Plummer and Meisler, Genomics 57, 323-331, 1999).

This polymorphism is potentially of more general importance because of the prominence of sodium channel blockade in the treatment of epilepsy (and other neurological conditions). For example, more than half of epilepsy patients treated pharmacologically in the UK receive a drug which principally targets the sodium channel (Shorvon et al., The Treatment of Epilepsy, Blackwell Publishers).

With respect to carbamazepine and phenytoin, this study provides a direction for a dosing scheme to be used in a prospective study to assess how pharmacogenetic diagnostics can improve dosing decisions. In particular, it may be clinically relevant to determine whether some individuals can safely be given more rapid dose increases. Although these polymorphisms explain relatively little of the total variation in the unselected cohort (6.5%, and 2.5% for phenytoin and carbamazepine, respectively), it is likely that in more controlled settings they will have a much larger proportionate effect. Our study did not take into account, for example, other AEDs taken together with phenytoin or carbamazepine, some of which are known to induce (e.g. phenobarbitone) or inhibit (e.g. sodium valproate) cytochrome P450 enzymes. Nevertheless, in our cohort we see average dose ranges across genotypes from 127 mg to 230 mg for phenytoin and carbamazepine respectively, indicating there is an important effect of these variants on dose. Presumably in a selected cohort the effect would be stronger. These results therefore suggest the possibility that genetic diagnostics could reduce the time it takes, on average, to control seizures using phenytoin and carbamazepine.

One limitation of the current study is that the database upon which our analyses were based records only the maximum dose received by each patient, rather than maintenance dose. The implications of this limitation are somewhat different for phenytoin and carbamazepine.

When the starting dose is insufficient to control seizures it may be increased until control is achieved. If the initial dose produces ADRs, on the other hand, it may be lowered. On balance, therefore, upward adjustment of dose is due to lack of efficacy and downward adjustment due to ADRs. This means, for example, that the effect of *3 on phenytoin dose is probably estimated conservatively here: any effect of *3 on dose reduction due to ADRs is not visible in our data. It would appear, however, that any effect of *3 on dose because of ADRs is small as we have observed no direct association between *3 and ADRs. For carbamazepine recording maximum dose seems less of a limitation as starting doses are virtually always less than what is finally necessary to control seizures.

Our results support the view that the major target, transporter, and drug metabolising enzyme are good starting points to study drug response and that pharmacogenetic traits are therefore more tractable for genetic analyses than those for common disease predisposition (Goldstein et al., 2003, supra). We also emphasize that a haplotype tagging strategy (Weale et al., 2003, supra) identified a previously unknown functional variant in the SCN1A gene. This functional variant was found 91 bp away from the nearest exon known at the time of the study, illustrating the need for exhaustive tagging.

Overall, our findings suggest that using genotype data may make it possible to safely reduce the time required to reach an effective dose. It is therefore also a priority to assess the utility of dose-adjustment on the basis of genotype for these medicines in a prospective clinical study. Prospective studies of carbamazepine and phenytoin, informed by a detailed retrospective study, would also serve as a useful model for future pharmacogenetic studies (Ingelman-Sundberg, Trends Pharmacol. Sci. 25, 193-200, 2004).

All publications and patent applications mentioned in this specification are indicative of the level of those skilled in the art to which this invention pertains.

All publications and patent applications are herein incorporated by reference to the same extent as if each individual publication or patent application was specifically and individually to be incorporated by reference.

Although the foregoing invention has been described in some detail by way of illustration and example for purposes of understanding, it will be clear to those skilled in the art that certain changes and modifications may be practiced within the scope of the appended claims.

1. A method for determining the dosage regime of a drug suitable for use in the treatment of a neurological condition in a subject which method comprises typing the SCN1A gene of the subject.

2. A method according to claim 1, wherein the typing comprises determining whether or not the subject has a polymorphism in the SCN1A gene associated with the response to the drug.

3. A method according to claim 2, wherein the polymorphism is a causative factor in the response to the drug or is in linkage disequilibrium with such a polymorphism.

4. A method according to claim 2, wherein the polymorphism is located in the 5′ splice donor site of the 5N alternatively spliced exon.

5. A method according to claim 2, wherein the typing comprises:
(i) determining the identity of the nucleotide at position 104 of SEQ ID NO: 1;
(ii) determining the identity of a nucleotide at a position corresponding to a nucleotide as defined in (i) in an allelic variant of SEQ ID NO: 1; or
(iii) determining the identity of a nucleotide in linkage disequilibrium with a nucleotide as defined in (i) or (ii).

6. A method according to claim 5, wherein the presence of A at position 104 of SEQ ID NO: 1, or at a corresponding position in an allelic variant thereof, is indicative of a subject requiring a higher maintenance dosage of the drug than a subject having G at the said position.

7. A method according to claim 5, wherein the identity of a nucleotide as defined in (i), (ii) or (iii) is determined for both alleles in the subject.

8. A method according to claim 7, wherein the absence or presence of A at position 104 of SEQ ID NO: 1, or at a corresponding position in an allelic variant thereof, is determined for both alleles in the subject.

9. A method according to claim 8, wherein the presence of A at position 104 of SEQ ID NO: 1, or at a corresponding position in an allelic variant thereof, at one or both alleles is indicative of a subject requiring a higher maintenance dosage of the drug than a subject having G at those alleles.

10. A method according to claim 9, wherein the presence of A at position 104 of SEQ ID NO: 1, or at a corresponding position in an allelic variant thereof, at both alleles is indicative of a subject requiring a higher maintenance dosage of the drug than a subject having G at one or both of those alleles.

11. A method according to claim 1, wherein the neurological condition is epilepsy, headache, chronic head or body pain syndrome, trigeminal neuralgia, manic depressive psychosis, a mood disorder or depression.

12. A method according to claim 1, wherein the drug is one which acts wholly or in part on the product encoded by the SCN1A gene.

13. A method according to claim 1, wherein the drug is an anti-epileptic drug (AED).

14. A method according to claim 13, wherein the AED is carbamazepine, phenytoin, lamotrigine, topiramate, oxcarbamazepine or valproate.

15. A method according to claim 1, which comprises determining whether or not the subject has a polymorphism in one or more additional genes associated with the response to the drug.

16. A method according to claim 15, wherein the one or more of the polymorphisms is a causative factor in the response to an AED or is in linkage disequilibrium with such a polymorphism.

17. A method according to claim 15, wherein the CYP2C9 gene is typed.

18. A method according to claim 17, wherein the typing comprises determining whether or not the subject carries the *3 allele.

19. A method according to claim 18, wherein the presence of one or two copies of the *3 allele is indicative of a subject requiring a lower maintenance dosage of the AED than a subject having fewer copies of the said allele.

20. A test kit suitable for use in a method for determining the dosage regime of a drug suitable for use in the treatment of a neurological condition in a subject, which test kit comprises means for typing the SCN1A gene of the subject.

21. A test kit according to claim 20, wherein the means for typing the SCN1A gene of the subject comprises:
(i) means for determining the identity of the nucleotide at position 104 of SEQ ID NO: 1;
(ii) means for determining the identity of a nucleotide at a position corresponding to a nucleotide as defined in (i) in an allelic variant of SEQ ID NO: 1; or
(iii) means for determining the identity of a nucleotide in linkage disequilibrium with a nucleotide as defined in (i) or (ii).

22. A test kit according to claim 21, wherein the means for determining the identity of the nucleotide at position 104 of SEQ ID NO: 1 comprises two oligonucleotides which can be used to amplify a polynucleotide comprising all or part of the sequence set out in SEQ ID NO: 1.

23. A test kit according to claim 22, wherein the means for determining the identity of the identity of a nucleotide at a position corresponding to a nucleotide as defined in (i) in an allelic variant of SEQ ID NO: 1 comprises two oligonucleotides which can be used to amplify a polynucleotide comprising all or part of an allelic variant of SEQ ID NO: 1.

24. A test kit according to claim 21, wherein the means for determining for determining the identity of a nucleotide in linkage disequilibrium with a nucleotide as defined in (i) or (ii) comprises two oligonucleotides which can be used to amplify a polynucleotide comprising all or part of the sequence set out in SEQ ID NO: 1 or an allelic variant thereof.

25. A test kit according to claim 24, wherein the two oligonucleotides have the sequences set out in SEQ ID NO: 5 and SEQ ID NO: 6.

26. A test kit according to claim 21, wherein the test kit further comprises the drug.

27. A test kit according to claim 21, wherein the neurological condition is epilepsy, headache, chronic head or body pain syndrome, trigeminal neuralgia, manic depressive psychosis, a mood disorder or depression.

28. A test kit according to claim 26, wherein the drug is one which acts wholly or in part on the product encoded by the SCN1A gene.

29. A test kit according to claim 26, wherein the drug is an anti-epileptic drug (AED).

30. A test kit according to claim 29, wherein the AED is carbamazepine, phenytoin, lamotrigine, topiramate, oxcarbamazepine or valproate.

31. A method for the treatment or prophylaxis of a neurological condition in a subject, which method comprises:
(a) determining the dosage regime of a drug suitable for use in the treatment of the neurological condition using a method according to claim 1; and
(b) administering a therapeutically effective amount of the drug to the subject in accordance with the dosage regime determined in (a).

32. A method according to claim 31, wherein the neurological condition is epilepsy, headache, chronic head or body pain syndrome, trigeminal neuralgia, manic depressive psychosis, a mood disorder or depression.

33. A method according to claim 31, wherein the drug is one which acts wholly or in part on the product encoded by the SCN1A gene.

34. A method according to claim 31, wherein the drug is an anti-epileptic drug (AED).

35. A method according to claim 34, wherein the AED is carbamazepine, phenytoin, lamotrigine, topiramate, oxcarbamazepine or valproate.

36. Products containing:
(i) means for typing the SCN1A gene of a subject; and
(ii) a drug suitable for use in the treatment of a neurological condition
as a combined preparation for simultaneous, separate or sequential use in a method of treatment of the human or animal body by therapy.


SEQUENCE LISTING (6 sequences)

SEQ ID NO: 1 - DNA, 109 bp, Homo sapiens
  CDS (9)..(98)
tctacaggtttgtaacagaatttgtaaacctaggcaatttttcagctcttcgcactttcagagtcttgagagctttgaaaactatttcggtaattccaggtaagaagtg

SEQ ID NO: 2 - protein, 30 aa, Homo sapiens
FVTEFVNLGNFSALRTFRVLRALKTISVIP

SEQ ID NO: 3 - DNA, 109 bp, Homo sapiens
  CDS (9)..(98)
tattcaggtacgtcacagagtttgtggacctgggcaatgtctcggcattgagaacattcagagttctccgagcattgaagacgatttcagtcattccaggtgagagcaa

SEQ ID NO: 4 - protein, 30 aa, Homo sapiens
YVTEFVDLGNVSALRTFRVLRALKTISVIP

SEQ ID NO: 5 - DNA, 20 bp, Artificial Sequence
  misc_feature: RT-PCR forward primer
ccacctctgccctgtacatt

SEQ ID NO: 6 - DNA, 20 bp, Artificial Sequence
  misc_feature: RT-PCR reverse primer
ctcccacaatggttttcagg